(19) United States
(12) Patent Application Publication
(10) Pub. No.: US 2011/0218605 A1
(43) Pub. Date: Sep. 8, 2011

(54) UPGRADEABLE IMPLANTABLE DEVICE

(76) Inventor: Adrian Cryer, New South Wales (AU)

(21) Appl. No.: 13/063,435

(22) PCT Filed: Sep. 10, 2009
(86) PCT No.: PCT/AU2009/001185

§ 371 Date: Mar. 10, 2011

(30) Foreign Application Priority Data
Sep. 10, 2008 (AU) 2008904715
Sep. 10, 2008 (AU) 2008904717

(51) Int. Cl. A61N 1/00

(52) U.S. Cl. 607/137

(57) ABSTRACT

An implantable device including an implantable first module having an elongate carrier member connected to a first housing; a stimulation unit disposed in the first housing and configured to utilize electrical signals received from a second module, and an electrically conductive first lead including at least one electrical conductor extending from the first housing at a proximal end of the first lead to a distal end of the first lead and an electrical insulator covering the at least one conductor at the proximal and distal ends of the first lead, wherein the first lead is configured to provide the signals from the second module to the stimulation unit when the first lead is electrically connected to the second module.

### CROSS-REFERENCE TO RELATED APPLICATIONS

This application is a National Stage Application of International Patent Application No. PCT/AU2009/001185, filed Sep. 10, 2009, which claims priority to Australian Provisional Patent Application No. 2008904717, filed on Sep. 10, 2008, and Australian Provisional Patent Application No. 2008904715, filed on Sep. 10, 2008, the contents of which is hereby incorporated by reference herein.

### BACKGROUND

1. Field of the Invention

The present invention is generally directed to an upgradeable implantable device, and more particularly, to an upgradeable implantable device.

2. Related Art

In many people who are profoundly deaf, the reason for deafness is absence of or destruction of, the hair cells in the cochlea which transduce acoustic signals into nerve impulses. These individuals are typically unable to derive sufficient benefit from conventional hearing aid systems because of damage to or absence of normal mechanisms for generating nerve impulses from.

Cochlear implant systems typically bypass the hair cells in the cochlea and deliver electrical stimulation to the auditory nerve fibers of a recipient directly, thereby allowing the brain to perceive a hearing sensation resembling the natural hearing sensation normally delivered to the auditory nerve.

Cochlear implant systems generally include an external component, which may include a sound processor, and an implantable component, which may include a receiver/stimulator unit. These components typically cooperate to provide a sound sensation to a recipient.

The external component has traditionally included a microphone for detecting sounds, such as speech and environmental sounds, a speech processor that converts the detected sounds into a coded signal, a power source, such as a battery, and an external transmitter antenna coil.

The coded signal output by the speech processor is transmitted transcutaneously to the implanted receiver/stimulator unit situated within a recess of the temporal bone of the recipient. This transcutaneous transmission occurs via the external transmitter antenna coil which is positioned to communicate with an implanted receiver antenna coil electrically connected to the receiver/stimulator unit. In this way both the coded sound signal and power may be provided transcutaneously to the implanted receiver/stimulator unit. In addition, the transcutaneous transmission may be performed using a radio frequency (RF) magnetic induction link.

The implanted receiver/stimulator unit can include a receiver antenna coil that receives the coded signal and power from the external processor component, and a stimulator that processes the coded signal and outputs a stimulation signal to an intra-cochlear electrode assembly which applies the electrical stimulation directly to the auditory nerve producing a hearing sensation corresponding to the original detected sound.

### SUMMARY

In one aspect of the present invention, an implantable device comprising an implantable first module is disclosed. The implantable first module comprises an elongate carrier member connected to a first housing and including a plurality of electrodes disposed at least partially on or in the carrier member, a stimulation unit disposed in the first housing and configured to utilize electrical signals received from a second module, and an electrically conductive first lead including at least one electrical conductor extending from the first housing at a proximal end of the first lead to a distal end of the first lead and an electrical insulator covering the at least one conductor at the proximal and distal ends of the first lead, wherein the first lead is configured to provide the signals from the second module to the stimulation unit when the first lead is electrically connected to the second module.

In another aspect of the present invention, an implantable device comprising an implantable first module is disclosed. The implantable first module comprises one or more electrically conductive wires having an antenna configuration and a lead configuration, and a receiver unit disposed in a first housing, electrically connected to the one or more wires, configured to process signals detected by the wires when the wires are in the antenna configuration and to utilize electrical signals received through the wires from a second module when the wires are in the lead configuration, wherein the wires are configured to electrically connect the receiver unit to a second module in the lead configuration.

In yet another aspect of the present invention, a method of electrically connecting implantable first and second modules of an implantable device is disclosed. The first module includes an electrically conductive first lead including at least one electrical conductor extending from a first housing at a proximal end of the first lead to a distal end of the first lead and an electrical insulator covering the at least one conductor at the proximal and distal ends of the first lead. The method comprises accessing said first module implanted in a recipient, electrically connecting, with an electrically conductive connector, a second lead of the second module and portion of the at least one conductor at the distal end of the first lead, providing, with the electrically connected first and second leads, electrical signals from the second module to the first module, and applying electrical stimulation to a recipient using the signals received from the second module.

In yet another aspect of the present invention, a method of electrically connecting implantable first and second modules of an implantable device is disclosed. The first module includes an antenna having one or more wires. The method comprises accessing the wires of the antenna, electrically connecting the second module to at least one of said one or more wires, and providing, with the one or more wires, electrical signals from the second module to the first module.

### BRIEF DESCRIPTION OF THE DRAWINGS

Embodiments of the present invention will now be described with reference to the accompanying drawings, in which:

FIG. 1 is a schematic diagram of an implantable component of a cochlear implant in accordance with embodiments of the present invention;

FIG. 2 is another schematic diagram of an implantable component of a cochlear implant in accordance with embodiments of the present invention;

FIG. 3 is a partial cross-sectional view of a lead of an implantable device in accordance with embodiments of the present invention;

FIGS. 4A to 4D are cross-sectional views of various conductor configurations for a lead of an implantable device in accordance with embodiments of the present invention;

FIGS. 5A to 5C are side views of a connector for leads of an implantable device in accordance with embodiments of the present invention;

FIG. 6 is a schematic diagram of an implantable component of a cochlear implant system in accordance with embodiments of the present invention; and

FIG. 7 is a schematic diagram of an implantable component having first and second modules in accordance with embodiments of the present invention.

### DETAILED DESCRIPTION

Embodiments of the present invention are illustrated and described herein as part of a cochlear implant system. It will be understood that embodiments of the present invention may also be implemented in other implantable devices and/or prostheses, including but not limited to auditory prostheses.

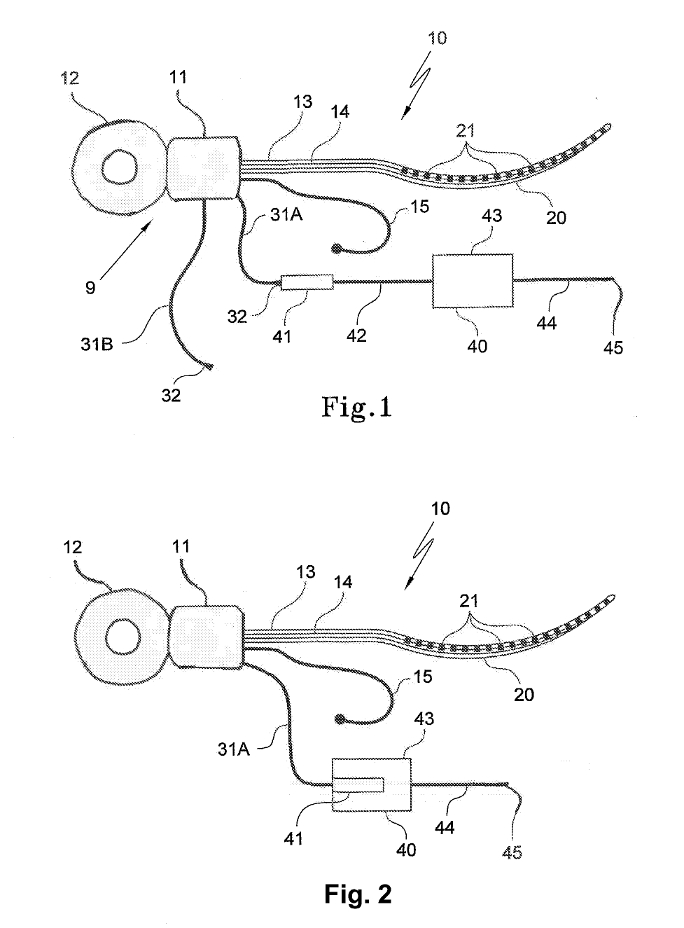

One example of an implantable component 10 of a cochlear implant system in accordance with embodiments of the present invention is illustrated in FIG. 1. The component 10 has a first stimulation module 9 which comprises a hermetically sealed and biocompatible titanium first housing 11. Extending from the first housing 11 is a cable 13 that extends to a carrier member 20 which has a plurality of electrodes 21 disposed thereon.

In the embodiment illustrated in FIG. 1, implantable component 10 has two electrically conductive leads 31A,31B extending from the first housing 11, with each lead 31a,31b having an end 32 distal the first housing 11 (referred to herein as “distal end 32”). In other embodiments, implantable component 10 may be have only one or more than two conductive leads 31. In one embodiment, the leads 31A,31B can be directly electrically connected to the componentry within the first housing 11. In another embodiment, the one or more leads can be isolated from the componentry within the first housing 11 by one or more galvanically isolated transformers or capacitors.

In one embodiment, the first housing 11 can be provided without powered and/or electronic componentry of the implantable component 10 and serves to act as an anchor member for a proximal end of the carrier member 20. The first housing 11 can have a feedthrough in the wall to provide electrical connection from the carrier member 20 to the interior of the first housing 11.

In the embodiment illustrated in FIG. 1, the first housing 11 can contain powered and/or electronic componentry. In the depicted embodiment, the first housing 11 contains a primary receiver/stimulator unit that receives signals detected by the antenna coil 12 and then decodes the signals and outputs signals suitable for delivery by the carrier member 20. The first housing 11 of the first stimulation module 9 can also house receiver circuitry for the antenna 12. For example, the first housing 11 can house a rectifier and decoding circuitry.

In other embodiment, the first housing 11 could instead just contain a stimulation unit that decodes incoming signals received from a second module 40 (as described below), or another module, and outputs signals suitable for delivery by the carrier member 20 to a neural network of the recipient of the cochlear implant system (sometimes referred to as an “implantee”). In embodiments in which the implantable device is a cochlear implant, the carrier member 20 can be insertable into a cochlea, for example the scala tympani of a cochlea. In such embodiments, antenna 12 may be absent from module 9 and could instead be mounted on the second module 40 or another module.

In certain embodiments, antenna coil 12, wherever it is positioned, can comprise one or more windings of a suitable electrically conductive material. The windings can extend from a further feedthrough formed in the outer wall of the housing of the module to which it is mounted. The windings can be formed from a suitable biocompatible material, such as platinum or gold, and/or be contained within an electrically insulating covering (or surround). In one embodiment, the covering can be formed of an elastomeric material, such as a silicone. A magnet can be disposed within the antenna coil. The use of the magnet within the antenna coil allows the antenna coil to be appropriately aligned with an external antenna coil to form a transcutaneous radio frequency (RF) magnetic induction link.

In yet a further embodiment, the first stimulation module 9 and carrier member 20 could be at least a portion of a totally implantable prosthesis, such as a totally implantable cochlear implant. In such embodiments, the implantable component 10 may include certain features that may enable the prosthesis to operate, at least temporarily, in a stand-alone fashion. For example, the component 10 could include a microphone, a speech processor, a power source, a power source controller and/or a power monitor. In such embodiments, the power source can comprise a rechargeable battery and so allow the implantable component to operate for a period of time without interaction with an external component. For example, the controller can control when power is delivered and the magnitude of the delivered voltage. The power monitor can monitor the operation of the power source and provide feedback to the controller. The power monitor can also provide an output that can be delivered to an external component to allow the recipient or a third party to determine at least some aspects of the operation of the power source.

In the embodiment illustrated in FIG. 1, first stimulation module 9 is designed to not be removed from the implantee following placement of the carrier member 20.

In the embodiment illustrated in FIG. 1, the implantable component 10 comprises at least one second module 40. The second module 40 is electrically connected to the first stimulation module 9 by a lead 31A, an electrical connector 41 and a cable 42. In this embodiment, the second module 40 is designed to be removable from the implantee if and when desired and/or at least relatively more readily removable than the first stimulation module 9.

The second module 40 has a second housing 43 containing powered and/or electronic componentry. This second housing 43 can also be hermetically sealed and be formed from a biocompatible material, such as titanium. The powered componentry of the second module 40 can comprise a secondary receiver/stimulator unit that outputs signals via the cable 42, electrical connector 41, and lead 31A to the first stimulation module 9 which in turn delivers the signals to the carrier member 20. The second module 40 can house a signal encoder, a driver circuit, and impedance matching and isolation circuitry. The housing 43 can also act as at least one electrode.

In other embodiments, the second module 40 can have an antenna (not shown) mounted thereon or extending therefrom. This antenna can also comprise an antenna coil and can have one, some or all of the features of other antenna coils described herein. The second module 40 can house receiver circuitry for the antenna. For example, the second module can house a rectifier and decoding circuitry.

In certain embodiments, the second module 40 can also house a power source. The power source can comprise a rechargeable battery. In some embodiments, the second module 40 can also house a power source controller that controls the operation of the power source and/or a power monitor. For example, the controller can control when power is delivered and the magnitude of the delivered voltage. The power monitor can monitor the operation of the power source and provide feedback to the controller. The power monitor can also provide an output that can be delivered to an external component to allow the recipient or a third party to determine at least some aspects of the operation of the power source.

In certain embodiments, the second module 40 can also house or support one or more microphone assemblies. Other possible componentry provided by the second module for the component 10 can comprise one or more of a temperature sensor, a humidity sensor, an impact or shock sensor, such as an accelerometer, and/or an optical communications or stimulation interface.

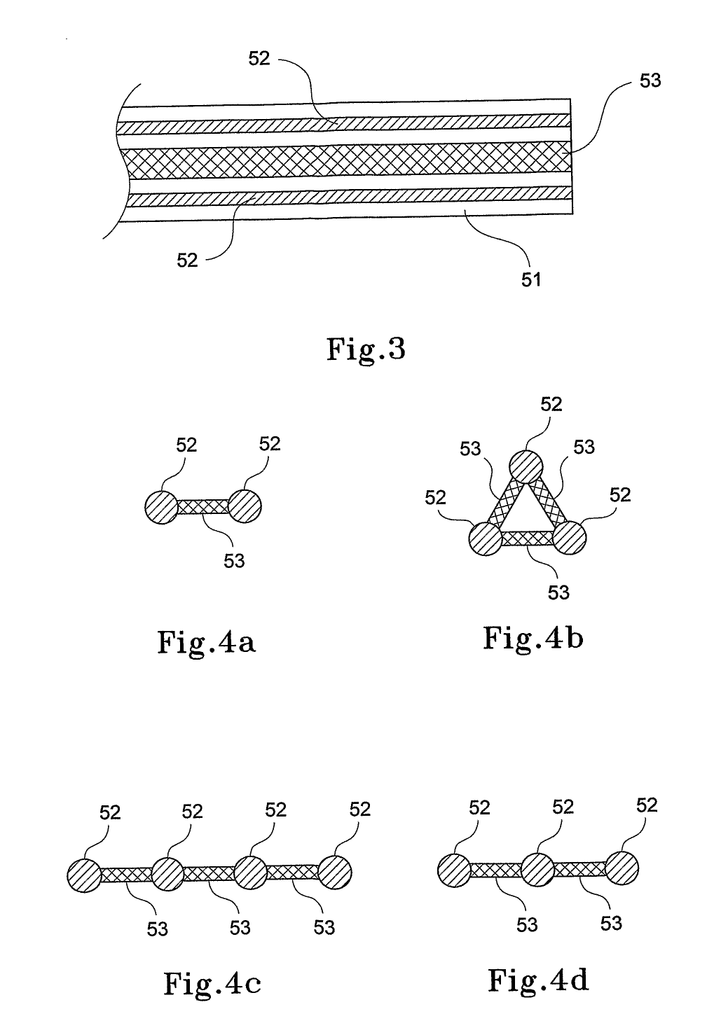

One or both leads 31A,31B can have the lead construction illustrated in FIGS. 3 to 4D. FIG. 3 depicts a portion of a lead at or adjacent its distal end 32. In the embodiment illustrated in FIG. 3, a lead, such as lead 31A or 31B, comprises an electrically insulating outer layer 51 surrounding two insulated electrical conductors 52 that extend through the lead from the housing 11 to the distal end 32.

In certain embodiments, the electrical conductor 52 can comprise a single wire, or can be include multiple strands. In the embodiment illustrated in FIG. 3, an elongate silicone mesh member 53 is disposed through the length of the lead and serves to separate the respective conductors 52 within the lead. In the embodiments illustrated in FIGS. 4A to 4D, a plurality of conductors 52 and mesh members 53 can be provided in the lead in various configurations, such as side-by-side and triangular configurations. Other embodiments may include other configurations.

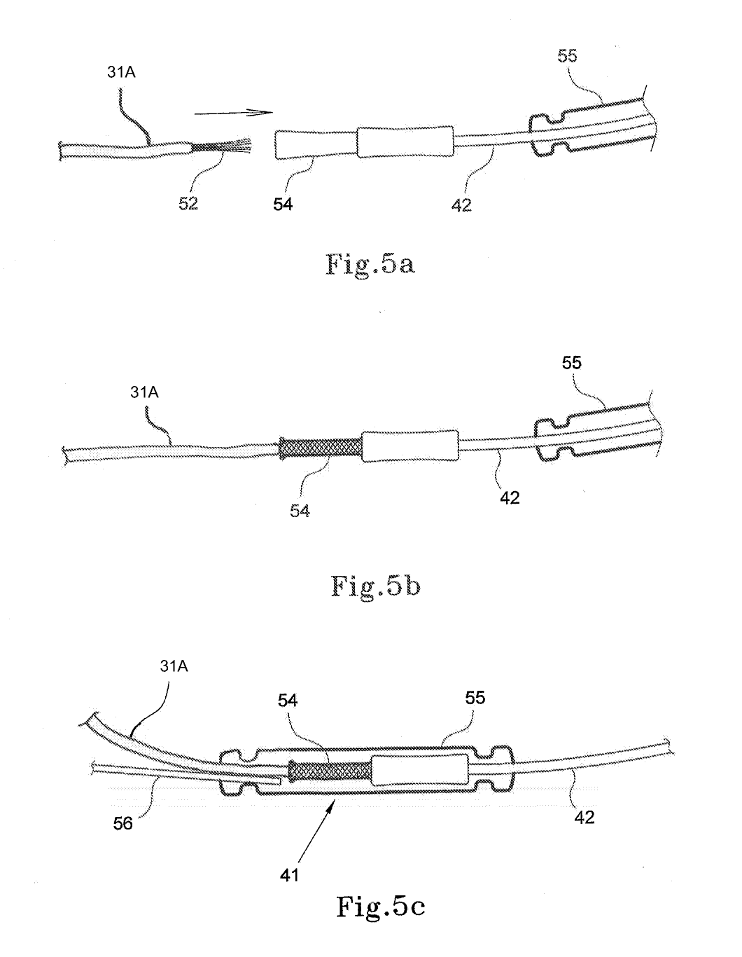

In certain embodiments, if desired, the lead can be cut or trimmed and then stripped to reveal the electrical conductor(s) 52. In the embodiment illustrated in FIGS. 5A to 5C, the electrically insulating outer layer can be firstly removed from lead 31A. The exposed conductor 52 can then be inserted into an electrically conductive joining member 54. In the depicted embodiment, the joining member comprises an electrically conductive and biocompatible tube member having at least one inner lumen that receives the distal end 32 of the exposed conductor 52. As depicted in FIG. 5B, the joining member 54 is then swaged or otherwise grips the conductor 52. In the depicted embodiment, the member 54 is formed of platinum but other suitable materials may be used in other embodiments.

As depicted in FIG. 5C, the connector 41 can further comprise a protective sleeve member 55. The sleeve member 55 can be slid along the lead 31A and/or cable 42 and over the joining member 54. The sleeve member 55 is fillable with a suitable electrically insulative material, for example a silicone, using a blunt needle 56.

In the embodiment illustrated in FIG. 1, the connector 41 can be located at a position between the first stimulation module 9 and the second module 40. As an example only, in certain embodiments, the connector can be approximately midway between the modules 9, 40. Irrespective of its location, the cable 42 can extend from the housing of the second module 40 to the connector 41.

In the embodiment illustrated in FIG. 2, the connector 41 can be mounted in the second housing 43 of the second module 40.

In other embodiments, the connector can comprise an insulation displacement connection in which a portion of the electrically insulating outer layer is stripped from the lead as a connector blade cuts through the outer layer to make contact with the electrical conductor. In alternative embodiments, connection can be achieved by crimping a connector onto the lead that is suitable for attachment to a receptacle on the second housing of the second module. In another embodiment, the connector may be an integral component of the second module.

In certain embodiments, implantable component 10 can comprise one or more leads 31B in addition to the lead 31a used to provide electrical connection with the second module 40. On initial implantation, said one or more leads can also have free distal ends 32, with the distal ends not necessarily being electrically connected to the second module or another module or implanted component, at least at the time of initial implantation of the implantable component 10. Rather, in certain embodiments, said one or more additional leads, being insulated, can be left in position under the skin. In such embodiments, the one or more additional leads are ready to be revealed during a subsequent surgery, trimmed if desired and then connected to a component as desired. In some embodiments, for example, such leads may be used when second module 40 is to be replaced and/or repaired.

Each of these additional leads 31B can have one, some or all of the features of said at least one lead 31A as described above.

In the embodiment illustrated in FIGS. 1 and 2, the second module 40 itself can have one or more electrically conductive leads 44 extending from the housing 43, each lead 44 having an end 45 distal the housing of the second module 40. The one or more leads 44 of the second module 40 can have one, some or all of the features described in relation to leads 31A, 31B. The lead 44 can be used to allow connection of one or more additional modules to the second module 40. In certain embodiments, this connection can be repeated to form a daisy chain of implantable modules. In such embodiments, the modules can serve as new modules that bypass and effectively replace the current module or can serve to allow addition of features to the totality of operation of the implantable component 10, as such features become available or become desired by the recipient.

In some embodiments, the carrier member 20 can comprise a non-insertable portion and an insertable portion. The non-insertable portion can comprise a proximal portion of the carrier member (e.g. cable 13) and have no electrodes disposed thereon. The insertable portion can comprise a portion of the carrier member extending back from the leading end of the carrier member. The insertable portion can have all of the electrodes 21 that are disposed on the carrier member. The carrier member 20 can decrease in diameter over at least a portion of its length towards the leading end. The carrier member 20 can be formed from an elastomeric material, such as a silicone. Each of the electrodes 21 can be formed from a biocompatible material, for example platinum. The electrodes 21 can comprise ring members. In one embodiment, the carrier member 20 can have 22 electrodes. In another embodiment, the carrier can have between about 20 and about 30 electrodes, 30 electrodes, or more than 30 electrodes. Extending from the first housing 11 via a feedthrough is a cable 13 that extends to an implantable tissue stimulating intracochlear electrode array 20 in the embodiment illustrated in FIG. 1. It will be noted that a series of wires 14 extend through the cable 13 to the plurality of electrodes 21. Not all of the wires 14 are depicted in FIG. 1 for reasons of clarity.

In the embodiment illustrated in FIG. 1, the implantable component 10 can have at least one secondary electrode assembly 15 extending from the first housing 11. The secondary electrode assembly 15 can have one or more electrodes. In embodiments in which the implantable component 10 is at least a portion of a cochlear implant, the electrode assembly 15 can be mounted external the cochlea of an recipient.

In one embodiment, the first housing 11 of the implantable component 10 can be positioned subcutaneously. In certain embodiments, first housing 11 may be positioned within a recess formed in the temporal bone of the recipient.

In some embodiments, the implantable component 10 can work in conjunction with an external component. The external component can be used to recharge the power source, where present, in either the first module 9 or second module 40. Still further, it can be used in conjunction with the implantable component 10 to provide a hearing sensation to a recipient. It will be appreciated that a different or the same external component can be used to recharge the power source and work in conjunction with the implantable component 10 to provide the hearing sensation. In one embodiment, the external component can have a microphone for detecting sound, a speech processor that converts the detected sounds, particularly speech, into a coded signal, a power source such as a battery, and an external transmitter antenna coil. The receiver/stimulator unit of the implantable component 10 and/or the second module 40 (or further modules if present) can receive the coded signal transmitted from the speech processor, process the coded signal and output a stimulation signal. The stimulation signal can be output to the carrier member 20. The carrier member 20 then delivers electrical stimulation to the auditory nerve of the recipient to produce a hearing sensation corresponding to the original detected sound. In some embodiments, the implantable component 10 may use input from the external component when it is present and rely on on-board componentry when the external component is not being used. In such embodiments, the implantable component 10 can be part of a partially or wholly implantable prosthesis.

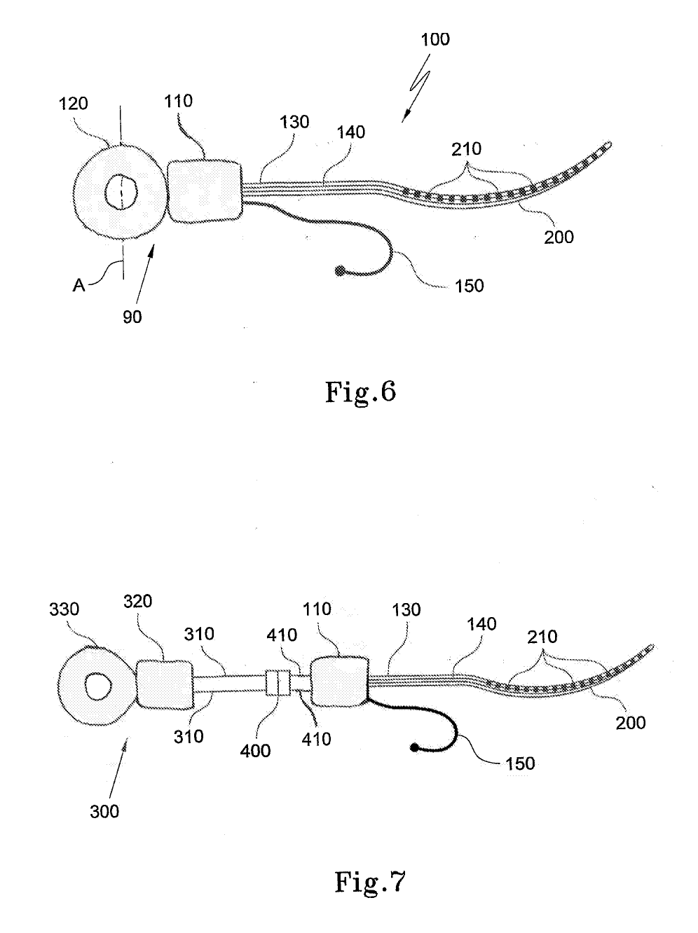

A method of modifying an implantable component of a prosthesis in accordance with embodiments of the present invention will now be described with reference to FIGS. 6 and 7. FIG. 6 depicts one example of an implantable component 100 that may be modified in accordance with embodiments of the present invention. In the embodiment illustrated in FIG. 6, the component 100 comprises a primary implantable module and has a primary receiver/stimulator unit 90 which comprises a hermetically sealed and biocompatible titanium housing 110. Extending from the housing 110 via a feedthrough is a cable 130 that extends to a carrier member 200 which has a plurality of electrodes 210 disposed thereon. The depicted carrier member 200 is insertable into a cochlea, for example the scala tympani of a cochlea.

In certain embodiments, in a first mode of operation, the primary receiver/stimulator unit 90 receives signals detected by a primary antenna 120 and then decodes the signals and outputs signals suitable for delivery to the recipient y the carrier member 200. In some embodiments, the housing 110 of the primary receiver/stimulator unit 90 can also house receiver circuitry for the primary antenna 120. For example, the housing 110 can house a rectifier and decoding circuitry.

In certain embodiments, the primary antenna 120 comprises one or more windings of a suitable electrically conductive material. The windings can extend from a further feedthrough formed in the outer wall of the housing 110 to which it is mounted. The windings can be formed from a suitable biocompatible material, such as platinum or gold, and/or are contained within an electrically insulating covering (or surround). In one embodiment, the covering can be formed of an elastomeric material, such as a silicone. A magnet can be disposed within the primary antenna 120. In such embodiments, the use of the magnet within the primary antenna 120 allows the antenna to be appropriately aligned with an external antenna coil to form a transcutaneous radio frequency (RF) magnetic induction link. In the embodiment illustrated in FIG. 6, the electrically conductive material included in antenna 120 is one or more electrically conductive wires (not shown). In the embodiment illustrated in FIG. 6, the one or more wires are arranged in an antenna configuration in which the wires are arranged as a coil (or coils) having one or more windings. As used herein, “antenna configuration” refers to an arrangement of wires in which the wires are configured to function as an antenna for receiving and/or transmitting signals wirelessly, such as through magnetic induction.

In one embodiment, the housing 110 of the implantable component 100 can be positioned subcutaneously. In some embodiments, housing 110 can be positioned within a recess formed in the temporal bone of the implantee. In certain embodiments, the primary receiver/stimulator unit 90 is designed to not be removed from the recipient following placement of the carrier member 200.

In certain embodiments, a process for modifying the implantable component 100 can comprise surgically exposing at least the primary antenna 120. In such embodiments, exposing the primary antenna 120 can comprise using a scalpel or other surgical tool to form an incision before peeling back the skin and body tissue, if present, that overlies the location of the primary antenna 120 in the recipient.

Once exposed, the wire coils that make up the primary antenna 120 can be accessed by the surgeon. In some embodiments, accessing the wire coils can comprise slicing the elastomeric support (along line A illustrated in FIG. 6, for example) that surrounds the wire coils. Prior to or during this step, the magnet, if present, can also be removed.

Once accessed, the wires that used to constitute the coils can be straightened and trimmed, if desired, to form the connecting wires 410 depicted in FIG. 7. In some embodiments, surgical scissors may be used to remove the elastomeric support from the wires or alternatively a surgical punch may be used. In the embodiment illustrated in FIG. 7, wires 410, which formerly formed at least part of the windings of antenna 120 (FIG. 6), are arranged in a lead configuration in which wires 410 are configured to electrically connect housing 110 with a portion of a connector 400. As used herein, “lead configuration” refers to an arrangement of wires in which the wires form at least a portion of a lead configured to electrically connect two or more devices, components, or other elements of a system.

In certain embodiments, various techniques can be employed to electrically connect the wires 410 to a further or secondary implantable module 300 illustrated in FIG. 7. In such embodiments, connecting the secondary implantable module can comprise forming an electrical connection between the wire or wires 410 that used to form the coils and the secondary implantable module 300. The electrical connection can be provided by electrically contacting the wires 410 to an electrical contact on the secondary implantable module 300. In the embodiment illustrated in FIG. 7, a connector 400 can be used to connect the wires 410 to the secondary implantable module 300.

In the embodiment illustrated in FIG. 7, leads 310 can also extend from the secondary implantable module 300 and may be connectable to the connector 400. In another embodiment, a suitable connector could be mounted in the housing 320 of the secondary implantable module 300.

In certain embodiments, the connector 400 can be formed using the technique as depicted in FIGS. 5A to 5C and described above.

In other embodiments, the connector 400 can comprise an insulation displacement connection in which a portion of the electrically insulating outer layer is stripped from the lead as a connector blade cuts through the outer layer to make contact with the electrical conductor. In alternative embodiments, connection can also be achieved by crimping a connector onto the lead that is suitable for attachment to a receptacle on the housing of the secondary implantable module. In another embodiment, the connector may be an integral component of the secondary implantable module.

In certain embodiments, the primary receiver/stimulator unit 90 and carrier member 200 may for at least a part of a partially or totally implantable prosthesis, such as a partially or totally implantable cochlear implant. In such embodiments, the implantable component 100 may include certain features that may enable the prosthesis to operate, at least temporarily, in a stand-alone fashion. For example, the component 100 could include a microphone, a speech processor, a power source, a power source controller and/or a power monitor. In such embodiments, the power source can comprise a rechargeable battery and so allow the implantable component to operate for a period of time without interaction with an external component. For example, the controller can control when power is delivered and the magnitude of the delivered voltage. The power monitor can monitor the operation of the power source and provide feedback to the controller. The power monitor can also provide an output that can be delivered to an external component to allow the recipient or a third party to determine at least some aspects of the operation of the power source. As described below, the power source controller and/or power monitor of the implantable component 100 can also be adapted to control and/or monitor, respectively, a power source present in the secondary implantable module 300. In another embodiment, the power source controller and/or power monitor can work in conjunction with similar devices also present in the secondary implantable module 300.

In certain embodiments, the carrier member 200 can comprise a non-insertable portion and an insertable portion. The non-insertable portion can comprise a proximal portion of the carrier member (e.g. cable 130) and have no electrodes disposed thereon. The insertable portion can comprise a portion of the carrier member extending back from the leading end of the member. The insertable portion can have all of the electrodes 210 that are disposed on the carrier member. The carrier member 200 can decrease in diameter over at least a portion of its length towards the leading end. The carrier member 200 can be formed from an elastomeric material, such as a silicone. Each of the electrodes 210 can be formed from a biocompatible material, for example platinum. The electrodes 210 can comprise ring members. In one embodiment, the carrier member 200 can have 22 electrodes. In another embodiment, the carrier can have between about 20 and about 30 electrodes, 30 electrodes, or more than 30 electrodes. A series of wires 140 extend through the cable 130 to the plurality of electrodes 210. Not all of the wires 140 are depicted in FIG. 6 for reasons of clarity.

In the embodiment illustrated in FIG. 6, the implantable component 100 can have at least one secondary electrode assembly 150 extending from the housing 110. The secondary electrode assembly 150 can have more one or more electrodes. In embodiments in which the implantable component is portion of a cochlear implant, the electrode assembly 150 can be mounted external the cochlea of the recipient.

In certain embodiments, the secondary implantable module 300 can be designed to be removable from the recipient or at least relatively more readily removable than the implantable component 100.

In some embodiments, the secondary implantable module 300 can be designed to work in conjunction with and/or supplement the primary receiver/stimulator unit 90 of the implantable component 100 once implanted. In such embodiments, the secondary implantable module 300 may take over control of the operation of the prosthesis. In other embodiments, the secondary implantable module 300, while adding functionality to the prosthesis, may still be controlled by the implantable component 100. In another embodiment, the secondary implantable module 300 can be designed to replace the function of the primary receiver/stimulator unit 90, particularly if the unit 90 has failed or is no longer suitable for the recipient.

In a further embodiment, the secondary implantable module 300 can also comprise a housing 320 containing powered and/or electronic componentry. This housing 320 can also be hermetically sealed and be formed from a biocompatible material, such as titanium. In certain embodiments, the powered componentry of the secondary implantable module 300 can comprise a secondary receiver/stimulator unit that outputs signals via the electrical connector 400 and the wires 410 that used to comprise the primary antenna 120 and into the housing 110 of the primary receiver/stimulator unit 90 which in turn delivers the signals to the electrode carrier member 200. In some embodiments, the secondary implantable module 300 can house a signal encoder, a driver circuit, and impedance matching and isolation circuitry.

In the embodiment illustrated in FIGS. 6 and 7, the secondary implantable module 300 can have a secondary antenna coil 330 mounted thereon. The secondary antenna 330 replaces the function of the primary antenna 120 of the implantable component 100 that is modified in embodiment illustrated in FIGS. 6 and 7. The secondary antenna 330 can have the same or different construction to that of primary antenna 120. In some embodiments, the secondary antenna 330 can also be appropriately aligned with an external antenna coil to form a transcutaneous radio frequency (RF) magnetic induction link. The secondary implantable module 300 can also house receiver circuitry for the secondary antenna 330. For example, the secondary implantable module 300 can house a rectifier and decoding circuitry.

In certain embodiments, the secondary implantable module 300 can house a power source regardless of whether the implantable component 100 has a power source or not. The power source can comprise a rechargeable battery.

In some embodiments, the secondary implantable module 300 can also house a power source controller that controls the operation of the power source and/or a power monitor. For example, the controller can control when power is delivered and the magnitude of the delivered voltage. The power monitor can monitor the operation of the power source and provide feedback to the controller. The power monitor can also provide an output that can be delivered to an external component to allow the recipient or a third party to determine at least some aspects of the operation of the power source. In certain embodiments, the power source controller and/or power monitor of the implantable component 100 can also be adapted to control and/or monitor, respectively, a power source present in the secondary implantable module 300. In another embodiment, the power source controller and/or power monitor of the implantable component 100 can work in conjunction with the power source controller and/or power monitor present in the implantable module 300.

In some embodiments, the secondary implantable module 300 can also house or support one or more microphone assemblies. In certain embodiment, other possible componentry of secondary implantable module 300 can comprise one or more of a temperature sensor, a humidity sensor, an impact or shock sensor, such as an accelerometer, and/or an optical communications or stimulation interface.

In certain embodiments, the implantable component 100 and secondary implantable module 300 can work in conjunction with an external component if and when desired. The external component can be used to recharge the power source, where present, in either the primary receiver/stimulator unit 90 or secondary implantable module 300. Still further, it can be used in conjunction with the implantable component 100 and/or secondary implantable module 300 to provide a hearing sensation to a recipient. It will be appreciated that in some embodiments a different or the same external component can be used to recharge the power source and work in conjunction with the implantable component 100 and/or secondary implantable module 300 to provide the hearing sensation. In one embodiment, the external component can have a microphone for detecting sound, a speech processor that converts the detected sounds, particularly speech, into a coded signal, a power source such as a battery, and an external transmitter antenna coil. The primary receiver/stimulator unit 90 of the implantable component 100 or the secondary implantable module 300 can receive the coded signal transmitted from the speech processor, process the coded signal and output a stimulation signal. The stimulation signal can be output to the carrier member 200. The carrier member 200 then delivers electrical stimulation to the auditory nerve of the recipient to produce a hearing sensation corresponding to the original detected sound. In some embodiment, the implantable component 100 and/or the secondary implantable module 30 may use input from the external component when it is present and rely on on-board componentry when the external component is not being used. In such embodiments, the primary and secondary implantable modules can work together to form a partially or fully implantable prosthesis, such as a fully or partially implantable cochlear implant system.

In certain embodiments, once electrical connection has been made between the housing 110 of the primary receiver/stimulator unit 90 and the housing 320 of the secondary implantable module 300, the incision site is surgically closed. Closure can be made using stitches, staples and/or adhesive.

The embodiment illustrated in FIGS. 6 and 7 provide a technique for modifying the implantable component 100 of a tissue-stimulating prosthesis, such as the primary receiver/stimulator 90 of a cochlear implant. In some embodiments, the modification technique can be performed when it is desired to upgrade or otherwise modify the performance of the implantable component 10 of the prosthesis. In certain embodiment, the technique may be used if the implantable component 100 has failed in some way. In some embodiments, the technique may provide a process for modifying or restoring operation of an at least partially implanted device without removing the electrode carrier member 200 from the cochlea. In certain embodiments, this technique may be beneficial for use with cochlear implants since it is desirable not to remove the intracochlear electrode array from its position within the cochlea once in place.

In certain embodiments, the technique can also be used with recipients that are children. For children under three years of age, it is currently considered preferable to not implant a totally implantable cochlear implant, as the recipient's head undergoes rapid growth during this period. As early implantation is also desirable to ensure an infant is capable of processing sounds, embodiments of the present invention provide the option of firstly implanting a traditional implantable component for the first few years of life. In such embodiments, the above-described technique may be performed at the appropriate time to place the secondary implantable module 300 in position within the recipient. In certain embodiments, this secondary implantable module 300 allows the recipient to potentially have the benefit of a totally implantable prosthesis without the risk of having to harm the relatively delicate structures of the cochlea of the recipient.

Further Embodiments

In certain embodiments, an implantable component of prosthesis comprises a first stimulation module comprising a first housing, at least one carrier member extending from the first housing to a leading end and having a plurality of electrodes disposed thereon, and one or more electrically conductive leads extending from the first housing, each lead having an end distal the first housing, wherein the lead comprises an electrically insulating outer layer surrounding one or more insulated electrical conductors extending through the lead from the first housing to the distal end.

In one embodiment, the implantable component can comprise one component of an auditory prosthesis. In one embodiment, the auditory prosthesis can comprise a cochlear implant. In one embodiment, the first housing has a hermetic sealed outer wall. The wall can be formed from a biocompatible material, such as titanium. In one embodiment, the first housing does not contain powered and/or electronic componentry for the implantable component and acts as an anchor member for a proximal end of the carrier member. The first housing can have a feedthrough in the wall to provide electrical connection from the carrier member to the interior of the first housing.

In a further embodiment, the first housing of the first stimulation module can contain powered and/or electronic componentry. In one embodiment, the first housing can contain a primary stimulator unit that decodes incoming signals and outputs signals suitable for delivery by the carrier member to a neural network of the implantee. In the case of a cochlear implant, the carrier member can be insertable into a cochlea, for example the scala tympani of a cochlea.

In another embodiment, the implantable component can have an antenna mounted thereon or extending therefrom. The antenna can comprise an antenna coil. The antenna coil can comprise one or more windings of a suitable electrically conductive material. The windings can extend from a further feedthrough formed in the outer wall of the first housing. The windings can be formed from a suitable biocompatible material, such as platinum or gold, and/or be contained within an electrically insulating surround. In one embodiment, the surround can be formed of an elastomeric material, such as a silicone. A magnet can be disposed within the antenna coil. The use of the magnet within the antenna coil allows the antenna coil to be appropriately aligned, with an external antenna coil to form a transcutaneous radio frequency (RF) magnetic induction link.

In some embodiments, the first housing of the first stimulation module can contain a primary receiver/stimulator unit that receives signals detected by the antenna and then decodes the signals and outputs signals suitable for delivery by the carrier member. The first housing of the first stimulation module can house receiver circuitry for the antenna. For example, the first stimulation module can house .a rectifier and decoding circuitry.

In yet a further embodiment, the implantable component can comprise a totally implantable prosthesis, such as a totally implantable cochlear implant. In such embodiments, the implantable component can have a microphone, a speech processor, a power source, a power source controller and/or a power monitor. The power source can comprise a rechargeable battery and so allow the implantable component to operate for a period of time separate from an external component. For example, the controller can control when power is delivered and the magnitude of the delivered voltage. The power monitor can watch the operation of the power source and provide feedback to the controller. The power monitor can also provide an output that can be delivered to an external component to allow the recipient or a third party to determine at least some aspects of the operation of the power source.

In certain embodiments, the first stimulation module can be designed to not be removable from the recipient following placement of the carrier member.

In a still further embodiment, the implantable component can comprise at least one second module. The second module can be electrically connected to the first stimulation module by at least one of said at least one leads and an electrical connector. In one embodiment, the second module can be designed to be removable from, the recipient if and when desired and/or at least relatively more readily removable than the first stimulation module.

In a further embodiment, the second module can comprise a second housing containing powered and/or electronic componentry. This second housing of the second module can also be hermetically sealed and be formed from a biocompatible material, such as titanium. In some embodiments, the powered componentry of the second module can comprise a secondary receiver/stimulator unit that outputs signals through the electrical connector and said at least one lead to the first stimulation module which in turn delivers the signals to the carrier member. The second module can house a signal encoder, a driver circuit, and impedance matching and isolation circuitry. In a still further embodiment, the second housing of the second module can act as at least one electrode.

In a further embodiment, the second module can have an antenna mounted thereon or extending therefrom. This antenna can also comprise an antenna coil. The antenna coil can comprise one or more windings of a suitable electrically conductive material. The windings can extend from a further feedthrough formed in the outer wall of the housing. The windings can be formed from a suitable biocompatible material, such as platinum or gold, and/or be contained within an electrically insulating surround. In one embodiment, the surround can be formed of an elastomeric material, such as a silicone. A magnet can be disposed within the antenna coil of the second module. In certain embodiments, the use of the magnet within this antenna coil allows the antenna coil to be appropriately aligned with an external antenna coil to form a transcutaneous radio frequency (RF) magnetic induction link. The second module can house receiver circuitry for the antenna. For example, the second module can house a rectifier and decoding circuitry.

In a still further embodiment, the second module can comprise a power source. The power source can comprise a rechargeable battery. The second module can also house a power source controller that controls the operation of the power source and/or a power monitor. For example, the controller can control when power is delivered and the magnitude of the delivered voltage. The power monitor can watch the operation of the power source and provide feedback to the controller. The power monitor can also provide an output that can be delivered to an external component to allow the recipient or a third party to determine at least some aspects of the operation of the power source.

In yet another embodiment, the second module can house or support one or more microphone assemblies. In other embodiments, other possible componentry of the second module can comprise one or more of a temperature sensor, a humidity sensor, an impact or shock sensor, such as an accelerometer, and/or an optical communications or stimulation interface.

In one embodiment, the connector can be located at a position between the first stimulation module and the second module. As an example only, the connector can be approximately midway between the modules. Irrespective of its location, a cable can extend from the second housing of the second module to the connector. In another embodiment, the connector can be mounted in the second housing of the second module.

In one embodiment, the one or more leads can be directly electrically connected to the componentry within the first housing. In another embodiment, the one or more leads can be isolated from the componentry within the first housing by one or more galvanically isolated transformers or capacitors.

In a further embodiment, one, some or each of the electrical conductors in the lead can comprise a single wire. In another embodiment, one, some or each of the electrical conductors can be comprised of multiple strands. An elongate silicone mesh member can be disposed through some or all of the length of the lead and serve to separate respective conductors within the lead. A plurality of such mesh members can be provided in the lead. In one embodiment, where two or more conductors are present, the conductors can be disposed in a side-by-side arrangement, with mesh members disposed therebetween. In other embodiment, other configurations may be used.

In a further embodiment, the connector can comprise an electrically conductive joining member. In one embodiment, the connector can comprise an electrically conductive tube member having at least one inner lumen that receives the distal end of said at least one conductor extending through the lead and which is then swaged or otherwise grips the conductor. The tube member can be formed of a biocompatible material, such as platinum.

In some embodiments, the connector can further comprise a protective sleeve member. The sleeve member can be slid along the lead and over the tube member. The sleeve member can be fillable with a suitable electrically insulative material, for example a silicone.

In another embodiment, the connector can comprise an insulation displacement connection in which a portion of the electrically insulating outer layer is stripped from the lead as a connector blade cuts through the outer layer to make contact with the electrical conductor. In another embodiment, connection can be achieved by crimping a connector onto the lead that is suitable for attachment to a receptacle on the housing of the second module. In another embodiment, the connector may comprise an integral component of the second module.

In certain embodiments, the implantable component can comprise one or more leads in addition to that used to provide electrical connection with the second module.

In some embodiments, on initial implantation, said one or more leads can also have free distal ends, with the distal ends not necessarily being electrically connected to the second module or another module or implanted component on at least initial implantation of the implantable component. Rather, said one or more additional leads, being insulated can be left in position under the skin. The one or more additional leads are, however, ready to be revealed during a subsequent surgery, trimmed if desired and then connected to a component, as desired. For example, such leads may be desired because the second module is to be replaced and/or repaired.

Each of these additional leads can have one, some or all of the features of said at least one lead as described above.

In yet another embodiment, the second module itself can have one or more electrically conductive leads extending from the second housing, each lead having an end distal the second housing. The one or more leads of the second module can comprise an electrically insulating outer layer surrounding one or more insulated electrical conductors extending through the lead from the second housing to the distal end. Such leads can be used to allow connection of a third module or higher number of modules to the second module. In some embodiments, this connection can be repeated to form a daisy chain of implantable modules. The modules can serve as new modules that bypass and effectively replace the current module or can serve to allow addition of features to the totality of operation of the implantable component, as such features become available or become desired by the recipient.

In certain embodiments, the carrier member can comprise a non-insertable portion and an insertable portion. The non-insertable portion can comprise a proximal portion of the carrier member and have no electrodes disposed thereon. The insertable portion can comprise a portion of the carrier member extending back from the leading end, of the member and is adapted to be inserted into the cochlea. The insertable portion can have all of the electrodes that are disposed on the carrier member. The carrier member can decrease in diameter over at least a portion of its length towards the leading end. The carrier member can be formed from an elastomeric material, such as a silicone. Each of the electrodes can be formed from a biocompatible material, for example platinum. The electrodes can comprise ring members. In one embodiment, the carrier member can have 22 electrodes. In another embodiment, the carrier can have between about 20 and about 30 electrodes, or 30 or more than 30 electrodes.

In another embodiment, the implantable component can have one or more secondary electrode assemblies extending from the housing. The secondary electrode assemblies can have more one or more electrodes. In the case of a cochlear implant, the one or more secondary electrode assemblies can be mounted within the cochlea of a recipient.

In one embodiment, the housing of the implantable component can be positioned subcutaneously. In some embodiments, the housing can be positioned within a recess formed in the temporal bone of the recipient.

In another embodiment, the prosthesis can have an external component. The external component can be used to recharge the power source. Still further, it can be used in conjunction with the implantable component to provide a hearing sensation to a recipient. In other embodiments, a different or the same external component can be used to recharge the power source and work in conjunction with the implantable component to provide the hearing sensation. In one embodiment, the external component can have a microphone for detecting sound, a speech processor that converts the detected sounds, particularly speech, into a coded signal, a power source such as a battery, and an external transmitter antenna coil. The receiver/stimulator unit of the implantable component and/or the second module (or further modules if used) can receive the coded signal transmitted from the speech processor, process the coded signal and output a stimulation signal. The stimulation signal can be output to an electrode assembly, such as an intracochlear electrode assembly. The electrode assembly then delivers electrical stimulation to the auditory nerve of the recipient to produce a hearing sensation corresponding to the original detected sound. The implantable component can work to use the input from the external component when it is present and rely on on-board componentry when the external component is not being used. In such embodiments, the implantable component can be part of a partially or wholly implantable prosthesis, such as a cochlear implant.

In other embodiments, an implantable component of a prosthesis comprises a first stimulation module comprising a first housing, at least one carrier member extending from the first housing to a leading end and having a plurality of electrodes disposed thereon, and one or more electrically conductive leads extending from the first housing, each lead having an end distal the first housing, wherein the lead comprises an electrically insulating outer layer surrounding one or more insulated electrical conductors extending through the lead from the first housing to the distal end, and at least one second module that is electrically connectable to the first stimulation module using at least one of said one or more electrically conductive leads.

In such embodiments, the implantable component, first stimulation module, second module, carrier member, and/or lead can have one, some or all of the features of the same component as defined herein with respect to other aspects and embodiments.

In such embodiments, for example, the second module can have a second housing which itself can have one or more electrically conductive leads extending therefrom, each lead having an end distal the second housing. The one or more leads of the second module can comprise an electrically insulating outer layer surrounding one or more insulated electrical conductors extending through the lead from the second housing to the distal end. Such leads can be used to allow connection of a third module or higher number of modules to the second module. In some embodiments, this connection can be repeated to form a daisy chain of implantable modules. In some embodiments, the modules can serve as new modules that bypass and effectively replace the current module or can serve to allow addition of features to the totality of operation of the implantable component, as such features become available or become desired by the recipient.

A method of modifying an implantable component of a tissue-stimulating prosthesis is also disclosed in accordance with embodiments of the present invention. In certain embodiments, the method is performed after the component has been implanted in a recipient, and the component has a primary implantable module comprising a primary receiver/stimulator unit and a primary antenna comprising one or more wires. In some embodiments, the method comprises surgically exposing at least the primary antenna, accessing the one or more wires, electrically connecting a secondary implantable module to at least one of said one or more wires.

In one embodiment, the tissue-stimulating prosthesis can comprise an auditory prosthesis. The auditory prosthesis can comprise a cochlear implant. The primary receiver/stimulator unit of the implantable component can have a housing having a hermetic sealed outer wall. The wall can be formed from a biocompatible material, such as titanium. In one embodiment, the primary receiver/stimulator unit decodes incoming signals and outputs signals suitable for delivery by an electrode carrier member to a neural network of the recipient. In embodiments in which the prosthesis is a cochlear implant, the electrode carrier member can comprise an intracochlear electrode array and be insertable into a cochlea, for example the scala tympani of a cochlea.

In some embodiments, the primary antenna can be electrically connected by a feedthrough to the primary receiver/stimulator unit within the housing. In one embodiment, the one or more wires of the antenna can comprise wire coils. The wires or wire coils can be formed from a suitable biocompatible material, such as platinum or gold, and/or be contained within an electrically insulating surround. In one embodiment, the surround can be formed of an elastomeric material, such as a silicone. A magnet can be disposed within the primary antenna. In certain embodiments, the use of the magnet within the primary antenna allows the primary antenna to be appropriately aligned with an external antenna, for example an external antenna coil, to form a transcutaneous radio frequency (RF) magnetic induction link.

In certain embodiments, prior to performing the method described herein, the primary receiver/stimulator unit receives signals detected by the primary antenna and then decodes the signals and outputs signals suitable for delivery by the electrode carrier member. The housing of the primary receiver/stimulator unit can also house receiver circuitry for the primary antenna. For example, the housing can house a rectifier and decoding circuitry.

In yet a further embodiment, the implantable component can comprise a partially or totally implantable prosthesis, such as a partially or totally implantable cochlear implant. For example, the primary implantable module can also comprise a power source and a power source controller. Still further, the primary implantable module can have a microphone, a speech processor, a power source, a power source controller and/or a power monitor. The power source can comprise a rechargeable battery and so allow the primary implantable module to operate for a period of time without interaction with an external component. For example, the power source controller can control when power is delivered and the magnitude of the delivered voltage. The power monitor can monitor the operation of the power source and provide feedback to the controller. The power monitor can also provide an output that can be delivered to an external component to allow the recipient or a third party to determine at least some aspects of the operation of the power source.

In one embodiment of the method according to the first aspect, the step of exposing the primary antenna can comprise using a scalpel or other surgical tool to form an incision before peeling back as desired the skin and body tissue, if present, that overlies the location of the, primary antenna. In some embodiments, the accessing the wires can comprise slicing the elastomeric surround surrounding the wire, removing the magnet, if present, and then removing the wire from the surround. In a further embodiment, and once accessed, the wires if in the form of coils can be straightened and trimmed, if desired. Surgical scissors may be used to remove the elastomeric surround or alternatively a surgical punch may be used.

In some embodiments, connecting the secondary implantable module can comprise forming an electrical connection between the wire or wires and the secondary implantable module. The electrical connection can be provided by electrically contacting the wire or wires to an electrical contact on the secondary implantable module. In a further embodiment, a connector can be used to connect the wire or wires to the secondary implantable module. In one embodiment, one or more leads can extend from the secondary implantable module and be connectable to said connector. In another embodiment, a suitable connector can be mounted in a housing of the secondary implantable module.

The secondary implantable module can be designed to be removable from the recipient if and when desired and/or at least relatively more readily removable than the housing of the primary implantable module of the implantable component.

In certain embodiments, the secondary implantable module can be designed to work in conjunction with and/or supplement the operation of the primary receiver/stimulator unit of the primary implantable module once implanted. In another embodiment, the secondary implantable module can be designed to replace the function of the primary receiver/stimulator unit, particularly if the unit has failed or is no longer suitable for the recipient.

In a further embodiment, the secondary implantable module can also comprise a housing containing powered and/or electronic componentry. This housing can also be hermetically sealed and be formed from a biocompatible material, such as titanium. In some embodiments, the powered componentry of the secondary implantable module can comprise a secondary receiver/stimulator unit that outputs signals through the electrical connector and the wiring that used to comprise the primary antenna and into the housing of the primary receiver/stimulator unit which in turn delivers the signals to the electrode carrier member. The secondary implantable module can house a signal encoder, a driver circuit, and/or impedance matching and isolation circuitry.

In a further embodiment, the secondary implantable module can have a secondary antenna mounted thereon or extending therefrom. In some embodiments, the secondary antenna can replace the function of the primary antenna of the primary implantable module that is modified in accordance with embodiments of the present invention. The secondary antenna of the secondary implantable module can also comprise an antenna coil. This antenna coil can comprise one or more windings of a suitable electrically conductive material. The windings can extend from a further feedthrough formed in the outer wall of the housing of the secondary implantable module. The windings can be formed from a suitable biocompatible material, such as platinum or gold, and/or be contained within an electrically insulating surround. In one embodiment, the surround can be formed of an elastomeric material, such as a silicone. A, magnet can be disposed within the secondary antenna coil. The use of the magnet within the secondary antenna coil allows the antenna coil to be appropriately aligned with an external antenna coil to form a transcutaneous radio frequency (RF) magnetic induction link. The implantable module can house receiver circuitry for the secondary antenna. For example, the secondary implantable module can house a rectifier and decoding circuitry.

In a still further embodiment, the secondary implantable module can comprise a power source. The power source can comprise a rechargeable battery. The secondary implantable module can also house a power source controller that controls the operation of the power source and/or a power monitor. For example, the controller can control when power is delivered and the magnitude of the delivered voltage. The power monitor can monitor the operation of the power source and provide feedback to the controller. The power monitor can also provide an output that can be delivered to an external component to allow the recipient or a third party to determine at least some aspects of the operation of the power source.

In yet another embodiment, the secondary implantable module can house or support one or more microphone assemblies. In certain embodiments, other possible componentry can comprise one or more of a temperature sensor, a humidity sensor, an impact or shock sensor, such as an accelerometer, and/or an optical communications or stimulation interface.

In one embodiment, the connector can be located at a position between the housing of the primary receiver/stimulator unit and the housing of the secondary implantable module. As an example only, the connector can be approximately midway between the primary receiver/stimulator unit and the secondary implantable module. In another embodiment, the connector can be mounted in the housing of the secondary implantable module.

In a further embodiment, the connector can comprise an electrically conductive joining member. In one embodiment, the connector can comprise an electrically conductive tube member having at least one inner lumen that receives the distal end of at least one conductor that extends through the lead and which is then swaged or otherwise grips the conductor. The tube member can be formed of a biocompatible material, such as platinum.

In some embodiments, the connector can further comprise a protective sleeve member. The sleeve member can be slid along the lead and over the tube member. The sleeve member can be finable with a suitable electrically insulative material, for example a silicone.

In another embodiment, the connector can comprise an insulation displacement connection which a portion of an electrically insulating outer layer of the lead is stripped from the lead as a connector blade cuts through the outer layer to make contact with the electrical conductor. In another embodiment, connection can be achieved by crimping a connector onto the lead that is suitable for attachment to a receptacle on the housing of the secondary implantable module. In another embodiment, the connector may comprise an integral component of the secondary implantable module.

The electrode carrier member can comprise a non-insertable portion and an insertable portion. The non-insertable portion can comprise a proximal portion of the carrier member and have no electrodes disposed thereon. The insertable portion can comprise a portion of the carrier member extending back from the leading end of the member and is adapted to be inserted into the cochlea, The insertable portion can have all of the electrodes that are disposed on the carrier member. The carrier member can decrease in diameter over at least a portion of its length towards the, leading end. The carrier member can be formed from an elastomeric material, such as a silicone. Each of the electrodes can be formed from a biocompatible material, for example platinum. The electrodes can comprise ring members. In one embodiment, the carrier member can have 22 electrodes. In another embodiment, the carrier can have between about 20 and about 30 electrodes, or 30 or more than 30 electrodes.

In another embodiment, the primary receiver/stimulator unit can have one or multiple secondary electrode assemblies extending from the housing. The secondary electrode assemblies can each have more one or more electrodes. In the case of a cochlear implant, the secondary electrode assembly can be mounted external the cochlea of a recipient.

In one embodiment, the housing of the primary receiver/stimulator unit can be positioned subcutaneously. In certain embodiments, the unit can be positioned within a recess formed in the temporal bone of the recipient.

In another embodiment, the prosthesis can have an external component. The external component can be used to recharge the power source that is part of the primary implantable module and/or the secondary implantable module, if present. Still further, it can be used in conjunction with the primary implantable module and/or the secondary implantable module, once implanted, to provide a hearing sensation to a recipient. It will be appreciated that a different or the same external component can be used to recharge the power source and work in conjunction with the primary implantable module and/or the secondary implantable module to provide the hearing sensation. In one embodiment, the external component can have a microphone for detecting sound, a speech processor that converts the detected sounds, particularly speech, into a coded signal, a power source such as a battery, and an external transmitter antenna coil. The primary receiver/stimulator unit of the primary implantable module or the secondary implantable module can receive the coded signal transmitted from the speech processor of the external component, process the coded signal and output a stimulation signal. The stimulation signal can be output to the carrier member. The carrier member then delivers electrical stimulation to the auditory nerve of the recipient to produce a hearing sensation corresponding to the original detected sound. In certain embodiments, the primary implantable module and/or the secondary implantable module can use input from the external component when it is present and rely on on-board componentry when the external component is not being used. In such embodiments, the primary and secondary implantable modules can work together to form a partially or fully implantable prosthesis, such as a cochlear implant system.

In certain embodiments, once electrical connection has been made between the housing of the primary receiver/stimulator unit and the housing of the secondary implantable module, the incision site is surgically closed. Closure can be made using stitches, staples and/or adhesive.

In some embodiments, electrically connecting an implantable module can be repeated, for example, by electrically connecting a tertiary implantable module to the wires constituting, for example, an antenna of the secondary implantable module. This can be repeated as desired with still further implantable modules. In another embodiment, the secondary implantable module can be removed or explanted and replaced with a tertiary implantable module. Again, this can be repeated as desired.

In another embodiment, an implantable component of a tissue-stimulating prosthesis as modified using the method as described herein.

In certain embodiments, a technique for modifying the implantable component of a tissue-stimulating prosthesis is provided. In some embodiments, the modification technique can be performed when it is desired to upgrade or otherwise modify the performance of the implantable component of the prosthesis. It can also be used if the implantable component has failed in some way. Certain embodiments of the present invention provides a technique for modifying or restoring operation without removing the electrode carrier member. In certain embodiments, this technique may be beneficial for use with cochlear implants since it is desirable not to remove the intracochlear electrode array from its position within the cochlea once in place.

In certain embodiments, the technique can also be used for recipients that are children. For children under three years of age, it is currently considered preferable to not implant a totally or fully implantable cochlear implant as the head undergoes rapid growth during this period. As early implantation is also desirable to ensure an infant is capable of processing sounds, embodiments of the present invention provide the option of firstly implanting one type of implantable component for the first few years of life. In such embodiments, the above-described technique may be performed at the appropriate time to place the secondary implantable module in position within the recipient. In certain embodiments, this secondary implantable module allows the recipient to potentially have the benefit of a totally or filly implantable prosthesis or a prosthesis with improved capability without the risk of having to harm the relatively delicate structures of the cochlea of the recipient.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents. The present embodiments are, therefore, to be considered in all respects as illustrative and not restrictive. Additionally, it will be appreciated that any features, components, elements, etc., described above in relation to different exemplary embodiments may be implemented together.

1-54. (canceled)

55. An implantable device comprising an implantable first module, the implantable first module comprising:
an elongate carrier member connected to a first housing and including a plurality of electrodes disposed at least partially on or in the carrier member;
a stimulation unit disposed in the first housing and configured to utilize electrical signals received from a second module; and
an electrically conductive first lead including at least one electrical conductor extending from the first housing at a proximal end of the first lead to a distal end of the first lead and an electrical insulator covering the at least one conductor at the proximal and distal ends of the first lead, wherein the first lead is configured to provide the signals from the second module to the stimulation unit when the first lead is electrically connected to the second module.

56. The device of claim 55, wherein the first module further comprises:
one or more electrically conductive second leads each including at least one electrical conductor extending from the first housing at a proximal end of the second lead to a distal end of the second lead and an electrical insulator covering the at least one conductor at the proximal and distal ends of the second lead.

57. The device of claim 55, wherein the first lead includes a plurality of conductors and one or more elongate silicone mesh members disposed between adjacent conductors for a length of the first lead.

58. The device of claim 57, wherein the conductors are disposed in a side-by-side within the electrical insulator of the first lead.

59. The device of claim 55, wherein the device comprises the second module.

60. The device of claim 59, wherein second module comprises a stimulation unit and the stimulation unit of the first module is configured to deliver to the carrier member one or more of the signals received from the second module.

61. The device of claim 59, wherein the stimulation unit is configured to decode the signals received from the second module and output stimulation signals to the carrier member for delivery to the recipient.

62. The device of claim 59, wherein the second module includes an electrically conductive first lead configured to be electrically connected to the first lead of the first module by an electrical connector.

63. The device of claim 59, wherein the second module comprises:
one or more electrically conductive second leads each including at least one electrical conductor extending from a second housing at a proximal end of the second lead to a distal end of the second lead and an electrical insulator covering the at least one conductor at the proximal and distal ends of the second lead.

64. The device of claim 55, wherein the device is a cochlear implant.

65. An implantable device comprising an implantable first module, the implantable first module comprising:
one or more electrically conductive wires having an antenna configuration and a lead configuration; and
a receiver unit disposed in a first housing, electrically connected to the one or more wires, configured to process signals detected by the wires when the wires are in the antenna configuration and to utilize electrical signals received through the wires from a second module when the wires are in the lead configuration, wherein the wires are configured to electrically connect the receiver unit to a second module in the lead configuration.

66. The device of claim 65, wherein the first module further comprises:
an elongate carrier member connected to a first housing and including a plurality of electrodes disposed at least partially on or in the carrier member.

67. The device of claim 66, wherein the device comprises the second module.

68. The device of claim 67, wherein the second module comprises an antenna.

69. The device of claim 67, wherein the second module comprises a power source.

70. The device of claim 67, wherein the second module comprises:
an electrically conductive lead including at least one electrical conductor extending from a second housing at a proximal end of the lead to a distal end of the lead and an electrical insulator covering the at least one conductor at the proximal and distal ends of the lead.

71. The device of claim 67, wherein the second module is configured to control the operation of the device when connected to the first module by the wires in the lead configuration.

72. The device of claim 67, wherein the second module is configured to be operated as an electrode of the device.

73. The device of claim 65, wherein the device is a component of an auditory prosthesis.

74. The device of claim 73, wherein the auditory prosthesis is a cochlear implant.

75. A method of electrically connecting implantable first and second modules of an implantable device, wherein the first module includes an electrically conductive first lead including at least one electrical conductor extending from a first housing at a proximal end of the first lead to a distal end of the first lead and an electrical insulator covering the at least one conductor at the proximal and distal ends of the first lead, the method comprising:
accessing said first module implanted in a recipient;
electrically connecting, with an electrically conductive connector, a second lead of the second module and portion of the at least one conductor at the distal end of the first lead;
providing, with the electrically connected first and second leads, electrical signals from the second module to the first module; and
applying electrical stimulation to a recipient using the signals received from the second module.

76. The method of claim 75, further comprising:
stripping the insulator from the distal end of the lead to expose a portion of the at least one conductor prior to said electrically connecting the first and second leads.

77. The method of claim 75, wherein the connector is an insulation displacement connector.

78. The method of claim 75, wherein the first module further comprises an elongate carrier member connected to the first housing and including a plurality of electrode disposed at least partially on or in the carrier member, and wherein said applying stimulation to the recipient comprises:
applying said electrical stimulation to the recipient with the carrier member.

79. The method of claim 78, wherein said applying stimulation to the recipient further comprises:
outputting, with the carrier member, provided from the second module to the first module with the electrically connected first and second leads.

80. A method of electrically connecting implantable first and second modules of an implantable device, wherein the first module includes an antenna having one or more wires, the method comprising:
accessing the wires of the antenna;
electrically connecting the second module to at least one of said one or more wires; and
providing, with the one or more wires, electrical signals from the second module to the first module.

81. The method of claim 80, wherein accessing the wires of the antenna includes cutting an elastomeric support in which the wires are disposed.

82. The method of claim 80, further comprising:
cutting at least one of the one or more wires prior to electrically connecting the second module to the at least one of said wires.

83. The method of claim 80, wherein accessing the wires of the antenna comprises:
surgically accessing the wires of the antenna when the antenna is implanted in a recipient.

84. The method of claim 80, wherein the first module further comprises a carrier member including a plurality of electrodes, and wherein the electrical signals are stimulation signals, the method further comprising:
providing the stimulation signals received from the second module to the carrier member.
